(19) United States
(12) Patent Application Publication
(10) Pub. No.: US 2010/0124517 A1
(43) Pub. Date: May 20, 2010

(54) Device for performing rapid 'on-site' tests of biological fluids

(75) Inventor: Lorenzo Cortelazzo, Padova (IT)

Correspondence Address: MODIANO & ASSOCIATI, Via Meravigli, 16, MILANO, 20123, IT

(73) Assignee: KALTEK S.R.L.

(21) Appl. No.: 12/591,231

(22) Filed: Nov. 13, 2009

(30) Foreign Application Priority Data
Nov. 19, 2008 (IT) PD2008A000338

(51) Int. Cl. G01N 33/48

(52) U.S. Cl. 422/58

(57) ABSTRACT

A device for performing rapid ‘on-site’ tests of biological fluids, comprising a box-like body adapted for accommodating and sealingly closing a holder of a stick of a rapid anti-doping test kit, the holder having an input hole for providing biological fluid towards the stick, the box-like body having externally at the input hole, a supporting collar adapted to accommodate and support an end of a cup-shaped tab of a container for biological fluids having a plurality of cup-shaped tabs, inside the collar elements for penetration of a bottom of the cup-shaped tab and elements for the passage of the biological fluid collected in the cup-shaped tab into the input hole of the holder of a stick being provided.

The present invention relates to a device for performing rapid ‘on-site’ tests of biological fluids.

### BACKGROUND OF THE INVENTION

Nowadays there is more and more a demand for rapid anti-doping or similar tests, not only in the sports field but also for evaluating physical changes of a driver, of a candidate of a public event, in occupational medicine, in the military area and many other fields.

The exams performed to create a Positive-Diagnosis, for example for a driver under elevated psychophysical condition, are structured in three levels.

At the first level there is the so-called rapid multitest of saliva or urine, also called screening test; the result delivered by this test is either a qualitative type of test or only indicate the presence or absence of narcotic substances in the organism of the examined person, and not the quantity of the consumed substance or still in circulation at the time of examination.

Such rapid tests are generally performed ‘on-site’, i.e. at the location where the person whose psychophysical condition has to be determined acts or has acted.

The second level is the exam in a laboratory, where also a quantitative result of the amount of a substance present in the organism of the examined person is determined and a positive result is defined with reference to a threshold value.

The third level relates to the execution of the confirmation exams with a chromatographic test, which has a medical-legal value.

The rapid screening tests are performed with non-invasive means which allow a collection of biological samples of saliva and urine in a relatively short time, in an economic, efficient and standardized way.

These tests allow to exclude the samples with negative results, i.e. the samples which do not contain narcotic substances or their metabolites, or the ones in which the concentration of such substances or metabolites is under a threshold value above which the results from an analytical test are considered positive and below are considered negative.

Nowadays, several kits are commercially available for performing tests instantaneously on site, i.e. they are usable at the location where the samples are taken, as, for example, the rapid urine screening test commercially known as SyvaRapidTest d.a.u. 4, a product from Syva Company Inc.

This rapid test allows for a qualitative determination of relative types of narcotics, such as cannabinoids, opioids, cocaine and methamphetamines in human urine.

This kit consists of a stick holder, flat-shaped, for accommodating a stick suitable to be moistened on one of its ends with a quantity of a biological fluid, for example urine, on such holder being provided an input hole for the drops of biological fluid at the lower end of the stick and an open window at the central region of the stick suitable to be colorized in one or more points in order to indicate the presence of one or more inappropriate substances contained in the drops of biological fluid poured into the input hole.

The kit comprises a pipette for taking the necessary drops of biological fluid from a separate container in which they are collected and for releasing them into the suitable hole on the stick holder.

Similar kits are well known, but whereas on the one hand their use is considered very useful, on the other hand they show disadvantages and inconvenience in their usage.

At first, the use of the pipette for drawing the biological fluid in a non-sterile environment may cause contamination of the biological fluid leading to false test results, not to count that in this way the biological fluid may easily be manipulated in a fraudulent manner on purpose.

Moreover, the holder containing the stick can be easily manipulated for manually changing the results, the stick being reachable, through the dedicated window on the holder containing it, with the tip of a pen or other instruments for adulterating the results.

For the collection of a biological fluid to be analyzed, it is today known, as well as described and claimed in EP0968766 in the name of this same applicant, a container for fluids, particularly for analysis of biological fluids, which is characterized in that it comprises a main body having a plurality of cup-shaped tabs for containment of such fluids, an intermediate element associatable with such body for closing it, provided with tubular portions insertable in such tabs, and an anti-tampering cover carrying a label is coupleable to such body.

The main body is provided with an open-top chamber, in which a user dispenses the required biological fluid, for example urine; the urine descends in the cup-shaped tabs, which open at the bottom of the open-top chamber.

The intermediate element is provided with as many tubular portions as the cup-shaped tabs, and its insertion into the open-top chamber causes the closure of the single cup-shaped tabs, so that they become as many independent containers containing biological fluids; the open-top chamber with the intermediate element therein is hermetically closed by means of the anti-tampering cover.

In a different embodiment, the intermediate element and the cover may be a single body.

The bottom of each cup-shaped tab bears a further protrusion in axial direction, suitable to be penetrated, i.e. broken by torsion or perforation, so that the biological fluid, and solely the biological fluid contained in the respective cup-shaped tab, can escape.

Such fluid containers, in particular for analyzing biological fluids, allow therefore with only one biological fluid sample having as many different and independent quantities of biological fluid as there are cup-shaped tabs, which protrude from the open-top chamber described above, each of which defines with the corresponding tubular portion for closing the intermediate element, an independent section.

Such containers are suitable for the use in laboratories, whereas their application for rapid on-site tests is not taken into consideration.

### SUMMARY OF THE INVENTION

The aim of the present invention is to provide a device for performing rapid ‘on-site’ tests of biological fluids, capable of combining the effectiveness of a kit for rapid on-site tests, for example for saliva or urine, with the safety and versatility of a container for biological fluids as described above.

Within this aim, an object of the invention is to provide a device which is easy to use, suitable to interface also with rapid test kits of a known type having containers for biological fluids, which may also be of a known type.

Another object of the invention is to provide a device which allows performing secure rapid tests, which can be alterated only by violating them clearly.

Another object of the invention is to provide a device for performing rapid ‘on-site’ tests of biological fluids, which is structurally simple and easy to use and which can be produced at low costs.

This aim, these objects and other which will become better apparent hereinafter are achieved by a device for performing rapid ‘on-site’ tests of biological fluids, characterized in that it comprises a box-like body suitable for accommodating and sealingly closing a stick holder of an anti-doping rapid test kit or the like, the holder having an input hole for providing biological fluid towards said stick, said box-like body having externally, in correspondence with said input hole, a supporting collar suitable to accommodate and support an end of a cup-shaped tab of a container for biological fluids having a plurality of cup-shaped tabs, inside said collar there being means for penetration of the bottom of said cup-shaped tab and means for the passage of biological fluid collected in said cup-shaped tab into said input hole of said stick holder.

### BRIEF DESCRIPTION OF THE DRAWINGS

Further characteristics and advantages of the invention will become better apparent from the following detailed description of a preferred but not exclusive embodiment of the device according to the invention, illustrated only by way of non-limitative example in the accompanying drawings, wherein:

FIG. 1 is a sectional side view of a device according to the invention;

FIG. 2 is a perspective view of the device according to the invention;

FIG. 3 is a sectional perspective view of the device according to the invention;

FIG. 4 is a partially sectional top view of the device according to the invention.

### DESCRIPTION OF THE PREFERRED EMBODIMENTS

With reference to the figures, a device 10 for performing rapid ‘on-site’ tests of biological fluids according to the invention comprises a box-like body 11, suitable for accommodating and sealingly closing a holder 12 of a stick 13 of a rapid anti-doping tests kit or the like, such as, for example, a rapid screening test kit of urine known commercially as Syva RapidTest d.a.u. 4, a product of Syva Company Inc.

The holder 12 has an input hole 14 for providing the biological fluid towards the stick 13.

The box-like body 11 has externally, at the input hole 14, a supporting collar 15 suitable to accommodate and support the end of a cup-shaped tab 16 of a container 17 for biological fluids.

The cup-shaped container 17, which can also be of a known type, is provided with a plurality of cup-shaped tabs, preferably four, 16, 16a, 16b, 16c, respectively.

Of the four cup-shaped tabs, the first one 16 is to be used with the biological fluid inside for a rapid test, the remaining three tabs 16a, 16b and 16c, which remain sealed, can be used at a later stage for analysis and counter-analysis in a laboratory.

Inside the collar 15 there are means for penetrating the bottom of the cup-shaped tab 16 and means for the passage of the biological fluid collected in the cup-shaped tab 16 in the input hole 14 of the holder 12 of the stick 13.

In this exemplary and non-limiting embodiment of device 10, the means for penetrating the bottom of the cup-shaped tab 16 are constituted by a needle 18.

The needle 18 acts also as means for the passage of the biological fluid from the tab 16, wherein it is contained, into the lower hole 14 of the holder 12 of the stick 13.

The needle 18 is arranged so as to protrude from the box-like body 11 inside the collar 15 so as to pass through the wall thickness of the box-like body 11.

The diameter of the needle 18 is such as to allow to dispense only a few drops of biological fluid into the hole 14.

The box-like body 11 consists of two half-shells, a first half-shell 11a and a second half-shell 11b, connected by a rear clamping hinge 19 and closed at the front with a snap-action device 20 comprising an extension tab 21 having a tooth latch 22 protruding from the half-shell 11b and a corresponding through opening with an edge 23 for engaging the tooth 22.

The snap-action device 20 guarantees that no change can be made to the test, which would require breaking the box-like body to access the stick 13.

Inside the box-like body 11, according to the embodiment described herein, centering means for correct positioning of the holder 12 in the box-like body 11 are provided, which protrude from the second half-shell 11b.

In this embodiment of the invention, such centering means are realized as a cross-protrusion 24, counter-profiled with respect to a recess 25 present in the bottom of the holder 12 of the stick 13.

The two half-shells 11a and 11b of the box-like body 11 may be realized in plastic material, molded as a single body, and then closed one on the other by folding the clamping hinge 19.

The box-like body 11 is made of transparent or semi-transparent plastic material which allows to see the results of the rapid test.

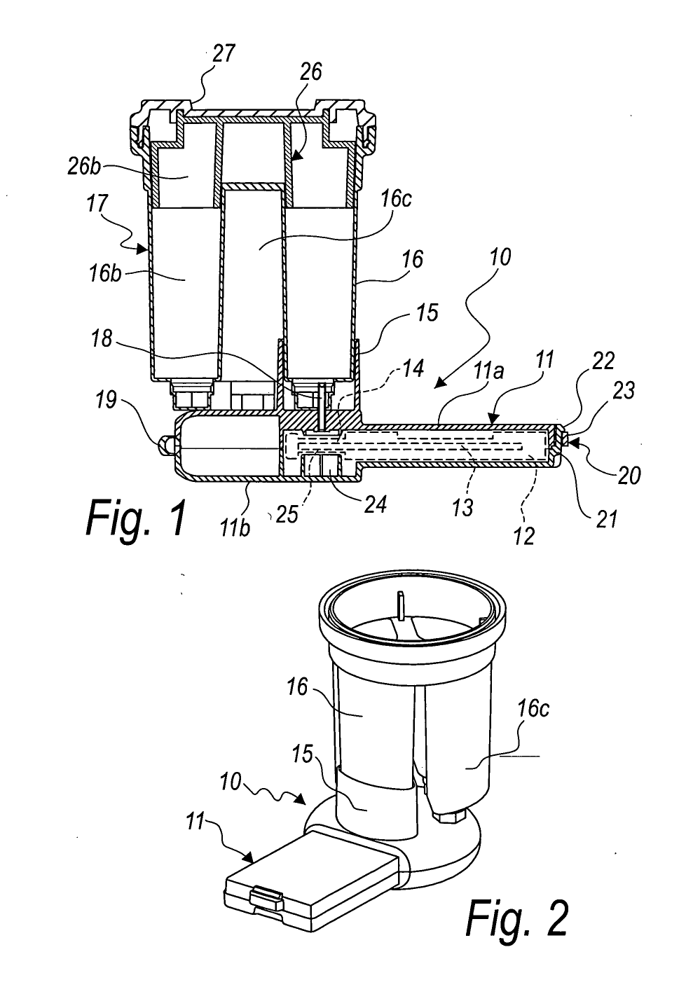

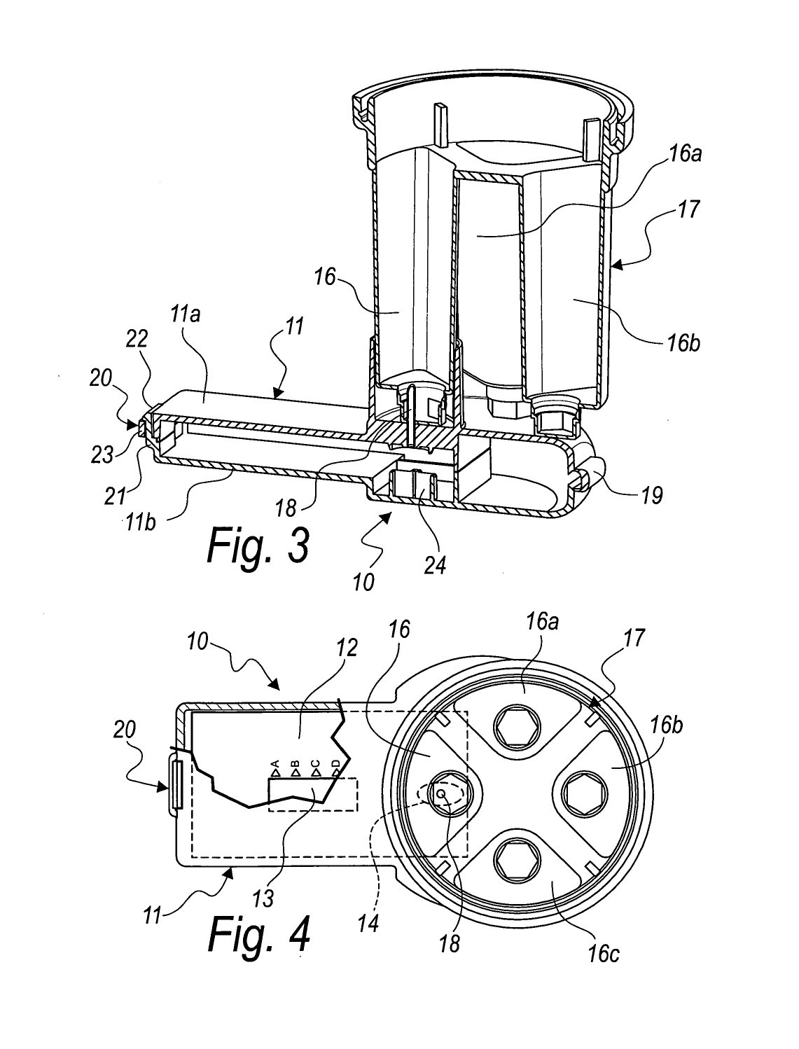

The container 17, as shown in sectional view in FIG. 1, comprises an intermediate element 26 having tubular portions 26, 26b for closing a corresponding cup-shaped tab 16, 16a, 16b, 16c and a label holder 27.

The intermediate element 26 and the holder 27 may be provided monolithically.

The use of the device 10 for performing rapid ‘on-site’ tests of biological fluids according to the invention is as follows.

The holder 12 of the stick 13 is placed in the second lower half-shell 11b of the box-like body 11, then the box-like body 11 is closed by closing as a book the first upper half-shell 11a on the lower half-shell 11b, thus providing the sealing snap closure.

The container 17, containing the biological fluid to be analyzed and separated in the four cup-shaped tabs 16, 16a, 16b, 16c is positioned so as to insert the first cup-shaped tab 16 in the collar 15 of the box-like body 11.

The box-like body 11 and the container 17 are pressed against each other so that the needle 18 perforates the bottom of the cup-shaped tab 16, causing the descent of the biological fluid in the hole 14 of the holder 12 of the stick 13.

The box-like body 11 and the container 17 are then sent together to the laboratory.

Alternatively, the box-like body 11 and the container 17 are separated; the dimensions of the hole on the bottom of the tab 16 are such that they prevent the exit of further biological fluid.

If the stick 13 signals the presence of particular substances, the container 17 is sent to a suitable laboratory for the necessary analysis.

In practice it has been observed that the invention achieves the intended aim and objects.

In particular, the invention provides a device for performing rapid ‘on-site’ tests of biological fluids, capable of combining the effectiveness of a rapid test kit, for example for saliva or urine, with the safety and versatility of a container for biological fluids as described above.

Moreover, the invention provides a device which is easy to use and is designed to interface also with known rapid test kits having containers for biological fluids which may also be of a known type.

Moreover, the present invention provides a device that allows performing rapid and safe tests, which can be altered only by violating clearly the box-like body 11 of the device 10.

Further, the invention provides a device for performing rapid ‘on-site’ tests of biological fluids, which is structurally simple and easy to use and can be produced with known means and at low costs.

The invention thus conceived is susceptible of numerous modifications and variations, all of which are within the scope of the appended claims; all the details may further be replaced with other technically equivalent elements.

In practice, the materials employed, as well as the contingent dimensions and shapes, may be any according to requirements and to the state of the art.

The disclosures in Italian Patent Application No. PD2008A000338 from which this application claims priority are incorporated herein by reference.

What is claimed is:

1. A device for performing rapid ‘on-site’ tests of biological fluids, comprising a box-like body adapted for accommodating and sealingly closing a holder of a stick of a rapid anti-doping test kit, the holder having an input hole for providing biological fluid towards said stick, said box-like body having externally at said input hole, a supporting collar adapted to accommodate and support an end of a cup-shaped tab of a container for biological fluids having a plurality of cup-shaped tabs, inside said collar means for penetration of a bottom of said cup-shaped tab and means for the passage of the biological fluid collected in said cup-shaped tab into said input hole of said holder of a stick being provided.

2. The device according to claim 1, wherein the means for penetrating the bottom of the cup-shaped tab are constituted by a needle.

3. The device according to claim 2, wherein said needle acts also as the means for the passage of the biological fluid from the cup-shaped tab into the hole at the holder of the stick.

4. The device according to claim 1, wherein said box-like body consists of two half-shells, a first half-shell and a second half-shell, connected by a rear clamping hinge and closed at a front with a snap-action device.

5. The device according to claim 1, wherein inside the box-like body means for correct positioning of the holder of the stick in the box-like body are provided.

6. The device according to claim 4, wherein the two half-shells of the box-like body are made of plastics, molded as a single body, and then closed one on the other by folding said rear clamping hinge.

7. The device according to claim 1, wherein the box-like body is made of transparent or semi-transparent plastics, at least in a region of the stick adapted to see the results of the rapid test.
